(19) United States
(12) Patent Application Publication
(10) Pub. No.: US 2025/0302543 A1
(43) Pub. Date: Oct. 2, 2025

(54) REGISTRATION OF IMAGING SYSTEM WITH SENSOR SYSTEM FOR INSTRUMENT NAVIGATION

(71) Applicant: AURIS HEALTH, INC., Santa Clara, CA (US)

(72) Inventors: Namita Anil Kumar, Redwood City, CA (US); Qasim Mahmood, Camarillo, CA (US); William Lanphier Chapin, Redwood City, CA (US); Chauncey F. Graetzel, Palo Alto, CA (US); Daniel Shalom Eliahu, Alamo, CA (US)

(73) Assignee: AURIS HEALTH, INC., Santa Clara, CA (US)

(21) Appl. No.: 19/006,086

(22) Filed: Dec. 30, 2024

Related U.S. Application Data
(60) Provisional application No. 63/571,768, filed on Mar. 29, 2024.

(51) Int. Cl. A61B 34/20; A61B 6/40; G06T 7/70; G16H 30/20

(52) U.S. Cl. CPC A61B 34/20; A61B 6/4085; G06T 7/70; G16H 30/20; A61B 2034/2051; A61B 2034/2068

(57) ABSTRACT

This disclosure provides methods, devices, and systems for planning and performing medical procedures. The present implementations more specifically relate to registering an image space with a sensor space for navigating an instrument within an anatomy. In some aspects, a controller for a medical system may determine a position of the instrument in the sensor space based on sensor data received from a sensor disposed on the instrument and may determine a position of the instrument in the image space based on image data captured by an imaging system external to the anatomy while the instrument is disposed within the anatomy. The controller determines a mapping between the image space and the sensor space based on the position of the instrument in the sensor space and the position of the instrument in the image space. In some implementations, the imaging system may be a cone beam computed tomography (CBCT) system.

### CROSS-REFERENCE TO RELATED APPLICATIONS

This application claims priority and benefit under 35 U.S.C. § 119 (c) to U.S. Provisional Patent Application No. 63/571,768, filed Mar. 29, 2024, which is incorporated herein by reference in its entirety.

### TECHNICAL FIELD

This disclosure relates generally to medical systems, and specifically to registration of an imaging system with a sensor system for instrument navigation.

### DESCRIPTION OF RELATED ART

Many medical procedures include steps that can be performed pre-operation (also referred to as a “preoperative phase”), intra-operation (also referred to as an “intraoperative phase”), or post-operation (also referred to as a “postoperative phase”). For example, during a preoperative phase, an imaging system may be used to scan or otherwise capture images or video of a patient's anatomy. Example suitable imaging technologies include computed tomography (CT), X-ray, fluoroscopy, positron emission tomography (PET), PET-CT, CT angiography, cone beam CT (CBCT), three-dimensional rotational angiography (3DRA), single-photon emission CT (SPECT), magnetic resonance imaging (MRI), optical coherence tomography (OCT), and ultrasound, among other examples. The images may be used, during an intraoperative phase, to help guide or navigate a medical instrument to a target (also referred to as a “treatment site”) within the patient's anatomy. However, images acquired during a preoperative phase may not accurately reflect a spatial relationship between the medical instrument and the target during an intraoperative phase. For example, among various other factors, preoperative images are often acquired several days (or even weeks) before the intraoperative phase, such that changes in the patient's anatomy may cause deviations in the spatial positioning of the target. Thus, there is a need to provide more accurate information about the spatial relationship between the medical instrument and the target during the intraoperative phase.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method for registering an image space with a sensor space. The method includes steps of receiving sensor data from a sensor disposed on an instrument within an anatomy; determining a position of the instrument in a first coordinate space based on the received sensor data; receiving image data captured by an imaging system external to the anatomy while the instrument is disposed within the anatomy; determining a position of the instrument in a second coordinate space based on the received image data; and determining a mapping between the first coordinate space and the second coordinate space based at least in part on the position of the instrument in the first coordinate space and the position of the instrument in the second coordinate space.

Another innovative aspect of the subject matter of this disclosure can be implemented in a controller for a medical system, including a processing system and a memory. The memory stores instructions that, when executed by the processing system, cause the controller to receive sensor data from a sensor disposed on an instrument within an anatomy; determine a position of the instrument in a first coordinate space based on the received sensor data; receive image data captured by an imaging system external to the anatomy while the instrument is disposed within the anatomy; determine a position of the instrument in a second coordinate space based on the received image data; and determine a mapping between the first coordinate space and the second coordinate space based at least in part on the position of the instrument in the first coordinate space and the position of the instrument in the second coordinate space.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows example components of the control system and the robotic system of FIG. 1, according to some implementations.

FIG. 3 shows a block diagram of an example localization system, according to some implementations.

FIG. 4 shows a block diagram of an example registration system, according to some implementations.

FIG. 5 shows another example medical system, according to some implementations.

FIG. 6 shows an example mapping between a computed tomography (CT) coordinate space and an electromagnetic (EM) coordinate space, according to some implementations.

FIG. 7 shows another example medical system, according to some implementations.

FIG. 8 shows another example medical system, according to some implementations.

FIG. 9 shows another example medical system, according to some implementations.

FIG. 10 shows an example medical instrument, according to some implementations.

FIG. 11 shows a block diagram of an example controller for a medical system, according to some implementations.

FIG. 12 shows an illustrative flowchart depicting an example operation for registering an image space with a sensor space, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures include a preoperative phase that precedes an intraoperative phase. During the preoperative phase, for some medical procedures, an imaging system may be used to scan or otherwise capture images or video of at least a portion of a patient's anatomy. For example, a computed tomography (CT) scanner may be used to acquire tomographic images (also referred to as “tomograms” or “CT scans”) of a patient's lungs during the preoperative phase for a bronchoscopy. A tomogram is a cross-section or slice of a three-dimensional (3D) volume. For example, multiple tomograms can be stacked or combined to recreate the 3D volume (such as a 3D model of the patient's lungs). Thus, tomograms can be used to detect a precise location or position (in 3D space) of a nodule or target in the patient's lungs. During the intraoperative phase, for some medical procedures, a medical system may use the preoperative images to generate a graphical interface for navigating a medical instrument within the patient's anatomy. For example, during a bronchoscopy, the medical system may detect a pose of an endoscope (such as a position and orientation of the scope in 3D space) based on sensor data received via an electromagnetic (EM) sensor disposed on the tip of the scope and map the pose of the endoscope to a 3D model of the patient's lungs depicted by the tomograms.

Accordingly, the graphical interface may depict a spatial relationship between the medical instrument and the target within the anatomy based on the sensor data and the image data. However, images acquired during a preoperative phase may not accurately reflect the spatial relationship between the medical instrument and the target during an intraoperative phase. For example, changes in the patient's anatomy or the medical environment can cause the spatial relationship between the endoscope and target to deviate from what is depicted by the graphical interface at any given time, which can lead to inaccurate navigation. Example factors include EM distortion, poor registration (or mapping) between the sensor space and the image space associated with the preoperative scans (also referred to as the “preoperative image space”), outdated preoperative scans, and anatomical deformations, among other examples. Aspects of the present disclosure recognize that some modern imaging technologies (such as cone beam CT) can be used to scan a patient's anatomy during an intraoperative phase. In some aspects, a medical system may capture updated images of the patient's anatomy during the intraoperative phase and use the updated image data to improve the representation of the spatial relationship between the medical instrument and the target. The updated image data and sensor data are often associated with different coordinate spaces. Thus, in some implementations, the medical system may “register” the updated image space with the sensor space to facilitate real-time navigation.

As used herein, the term “registration” refers to a mapping or transformation between different coordinate spaces. For example, a medical system (or registration system associated therewith) may register an imaging system used for capturing images of a patient's anatomy (such as a cone beam CT scanner) with a sensor system used for tracking a pose of a medical instrument within the anatomy (such as an EM field generator) by determining a mapping or spatial transformation that maps any point or vector in the image space to a respective point or vector in the sensor space (such as a transformation matrix). The terms “mapping,” “transformation,” “spatial transformation,” and “registration matrix,” may be used interchangeably herein. The terms “respective” and “corresponding” also may be used interchangeably herein.

Although certain aspects of the present disclosure are described in detail herein in the context of bronchoscopy, it should be understood that the systems and techniques of the present disclosure may be applicable to any medical procedure. Example medical procedures may include minimally invasive procedures (such as laparoscopy), non-invasive procedures (such as endoscopy), therapeutic procedures, diagnostic procedures, percutaneous procedures, and non-percutaneous procedures, among other examples. Example endoscopic procedures include bronchoscopy, ureteroscopy, gastroscopy, nephroscopy, and nephrolithotomy, among other examples. The terms “scope,” “endoscope,” “catheter,” and “instrument” may be used interchangeably herein.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples).

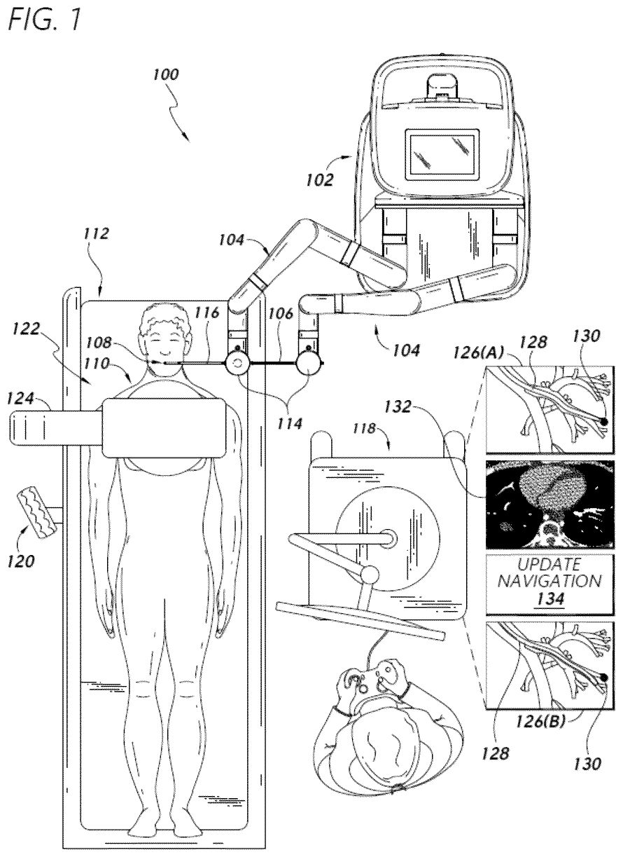

FIG. 1 shows an example medical system 100 (also referred to as a “surgical medical system” or a “robotic medical system”), according to some implementations. As shown in FIG. 1, the medical system 100 may be arranged for diagnostic or therapeutic bronchoscopy. The medical system 100 can include and utilize a robotic system 102 which can be implemented, for example, as a robotic cart. Although the medical system 100 is shown as including various cart-based systems or devices, the concepts disclosed herein can be implemented in any type of robotic system or arrangement, such as robotic systems employing rail-based components, table-based robotic end-effectors, or manipulators, among other examples. The robotic system 102 may include one or more robotic arms 104 (also referred to as “robotic positioners”) configured to position or otherwise manipulate a medical instrument 106 (such as a steerable endoscope or another elongate instrument). For example, the medical instrument 106 can be advanced through a natural orifice access point (such as the mouth 108 of a patient 110 positioned on a table 112) to deliver diagnostic or therapeutic treatment. Although described in the context of a bronchoscopy procedure, the medical system 100 also may be used to perform other types of medical procedures. Example suitable procedures include gastro-intestinal (GI) procedures, renal procedures, urological procedures, and nephrological procedures, among other examples.

With the robotic system 102 properly positioned, the medical instrument 106 can be inserted into the patient 110 robotically, manually, or a combination thereof. For example, the one or more robotic arms 104, or instrument drivers 114 coupled thereto, can control the medical instrument 106. In some implementations, the medical instrument 106 may be advanced within a sheath 116. For example, the sheath 116 may be coupled to, or controlled by, a robotic arm 104. In some implementations, the medical instrument 106 and the sheath 116 may each be coupled to a respective instrument driver from a set of instrument drivers 114. The instrument drivers 114 can be repositionable in space by manipulating the one or more robotic arms 104 into different angles or positions.

In the example of FIG. 1, the medical instrument 106 can be directed down the patient's trachea and lungs after insertion or advanced to a target destination or operative site. In some implementations, to enhance navigation through the patient's lung network or reach the desired target, the medical instrument 106 may be manipulated to telescopically extend from the outer sheath 116 to obtain enhanced articulation or greater bend radius. The use of separate instrument drivers 114 can allow the medical instrument 106 and sheath 116 to be driven independently of each other.

In some implementations, the medical instrument 106 may include an elongate member or shaft configured to be inserted or retracted, articulated, or otherwise moved within the anatomy. Further, in some implementations, the medical instrument 106 may include one or more imaging devices (such as cameras) positioned on a distal end of the elongate shaft or deployed through a working channel of the elongate shaft. The imaging devices can be configured to generate or capture image (or video) data or send the image data to another device or component. In some implementations, the medical instrument 106 may include an instrument base or one or more handles positioned at a proximal end of the medical instrument 106. The instrument base can be coupled to a manipulator (such as an end of a robotic arm 104). The instrument base can include one or more drive inputs coupled to one or more drive outputs of the manipulator, wherein the drive inputs or drive outputs act as an interface.

In some implementations, the medical instrument 106 may include a working channel configured to receive one or more other instruments or elements therein or provide other functionality. The working channel can extend axially, such as along the length of the medical instrument 106. Furthermore, the medical instrument 106 can include or be associated with one or more elongate movement members (such as pulls wires) that can extend from a proximal end through the elongate shaft to the distal end of the elongate shaft. The elongate movement members can be manipulated, such as by manipulators on the one or more robotic arms 104, to control actuation of the elongate movement members.

In some implementations, the medical instrument 106 may include one or more sensors, such as electromagnetic (EM) sensors, shape sensors (such as shape sensing fiber), accelerometers, gyroscopes, satellite-based positioning sensors (such as global positioning system (GPS) sensors), or radio-frequency (RF) transceivers, among other examples. The sensors can be configured to generate or produce sensor data or provide the sensor data to another device or component. The sensors can be disposed at a distal end of the elongate shaft or along a length of the elongate shaft. In some implementations, the medical instrument 106 may be configured to receive an elongate member or device through a working channel, wherein the elongate member includes one or more sensors along a length of the elongate member. One or more sensors on the medical instrument 106 may provide sensor data to control circuitry of the medical system 100, which is then used to determine a position, orientation, or shape of the medical instrument 106.

The medical system 100 can also include a control system 118 (also referred to as a “control tower” or “mobile tower”). The control system 118 can be communicatively coupled (such as via wired or wireless connections) to the robotic system 102 to control various aspects of the robotic system 102 (such as electronics, optics, sensors, or power) or one or more subsystems associated with the robotic system 102, such as a fluid management system (not shown). Placing such functionality in the control system 118 can allow for a smaller form factor of the robotic system 102 that may be more easily adjusted or re-positioned by an operator or user. Additionally, the division of functionality between the robotic system 102 and the control system 118 can reduce operating room clutter and facilitate efficient clinical workflow.

The medical system 100 can include an electromagnetic (EM) field generator 120, which is configured to broadcast or emit an EM field that can be detected by various EM sensors, such as a sensor disposed on the medical instrument 106. The EM field can induce small electric currents in coils of the EM sensors, which can be analyzed to determine a position, angle, or orientation of the EM sensors relative to the EM field generator 120. Although EM fields and EM sensors are described in many examples herein, position sensing systems or sensors can include various other types of position sensing systems or sensors, such as optical position sensing systems or sensors, image-based position sensing systems or sensors, among other examples.

The medical system 100 can further include an imaging system 122 (also referred to as an “imaging device”) configured to generate, provide, or send image data (also referred to as “images”) to another device or system. For example, the imaging system 122 can generate image data depicting an anatomy of the patient 110 and provide the image data to the control system 118, the robotic system 102, or another device. The imaging system 122 may include an emitter or energy source (such as an X-ray source) or a detector (such as an X-ray detector) mounted on a C-shaped arm support 124, which allows for flexibility in positioning around the patient 110 to capture images from various angles without moving the patient 110. Use of the imaging system 122 can provide visualization of internal structures or anatomy, which can be used for a variety of purposes, including navigation of the medical instrument 106 (such as by providing images of internal anatomy to a user) and localization of the medical instrument 106 (based on an analysis of image data), among other examples. In some aspects, the imaging system 122 may enhance the efficacy or safety of a medical procedure, such as a bronchoscopy, by providing clear, continuous visual feedback to the operating surgeon or team.

In some implementations, the imaging system 122 may be a mobile device configured to move around an environment. For example, the imaging system 122 can be positioned next to the patient 110 (as shown in FIG. 1) during a particular phase of a procedure and removed when the imaging system 122 is no longer needed. In some other implementations, the imaging system 122 may be part of the table 112 or other equipment in an operating environment. The imaging system 122 can be implemented as a Computed Tomography (CT) machine or system, X-ray machine or system, fluoroscopy machine or system, Positron Emission Tomography (PET) machine or system, PET-CT machine or system, CT angiography machine or system, Cone-Beam CT (CBCT) machine or system, three-dimensional rotational angiography (3DRA) machine or system, single-photon emission computed tomography (SPECT) machine or system, Magnetic Resonance Imaging (MRI) machine or system, Optical Coherence Tomography (OCT) machine or system, or ultrasound machine or system, among other examples. In some implementations, the medical system 100 may include different types of imaging systems that can be used or positioned over the patient 110 during different phases or portions of a procedure depending on the needs at that time.

In some implementations, the imaging system 122 may be configured to process multiple images (also referred to as “image data”) to generate a three-dimensional (3D) view or model. For example, the imaging device 122 can be implemented as a CT machine configured to capture or generate a series of images (also referred to as “tomograms) or image data representing two-dimensional (2D) cross-sections or slices of a 3D volume from different angles around the patient 110, and then use one or more algorithms to reconstruct these images or image data into a 3D model. The 3D model can be provided to the control system 118, robotic system 102, or another device, such as for processing or display.

In some implementations, image data from the imaging system 122 may be used to localize various elements, such as the medical instrument 106, a target within the anatomy, or specific anatomical features, among other examples. For example, the control system 118 can be configured to provide navigation information during a procedure to assist a user navigating the medical instrument 106 within the anatomy to reach a target (such as a desired treatment site or location). In some implementations, a target can include a nodule, such as in the context of certain bronchoscopy procedures. To illustrate, the control system 118 can display a navigation view or graphical data 126 that includes an instrument indicator 128 representing the medical instrument 106, a target indicator 130 representing the target, and an anatomical map. The navigation data 126(A) (such as initial navigation data) can be determined based on sensor data from a sensor of the medical instrument 106 (such as EM sensor data associated with the EM field generator 120), a map of the anatomy, or a location of the target. In some implementations, the map or location of the target may be determined based on preoperative data, such as data obtained during a preoperative procedure to find a target location or map the anatomy.

In some implementations, the navigation data 126(A) may be dynamically updated based on image data 132 from the imaging system 122. For example, the control system 118 can receive the image data 132 and analyze the image data 132 to determine a current or actual spatial relationship between the medical instrument 106 and the target. In some implementations, the control system 118 may display the image data 132 to a user, receive user input indicating a position of the medical instrument 106 or a position of the target in the image data 132, and analyze the image data 132 based on the user input to determine the current spatial relationship. If the control system 118 determines that the navigation data 126(A) incorrectly depicts the location of the medical instrument 106 (such as where the spatial relationship associated with the image data 132 is different than the spatial relationship associated with the navigation data 126(A)), the control system 118 may update the navigation data 126(A) at 134 and provide updated navigation data 126(B) that reflects the current or near real-time position of the medical instrument 106 relative to the target or the map.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, the Internet, personal area networks (PANs), body area network (BANs), etc. In some examples, various communication interfaces can include wireless technology, such as Bluetooth, Wi-Fi, near-field communication (NFC), or the like. Furthermore, in some examples, the various components of the medical system 100 can be connected for data communication, fluid exchange, power exchange, and so on, via one or more support cables, tubes, connections, or the like.

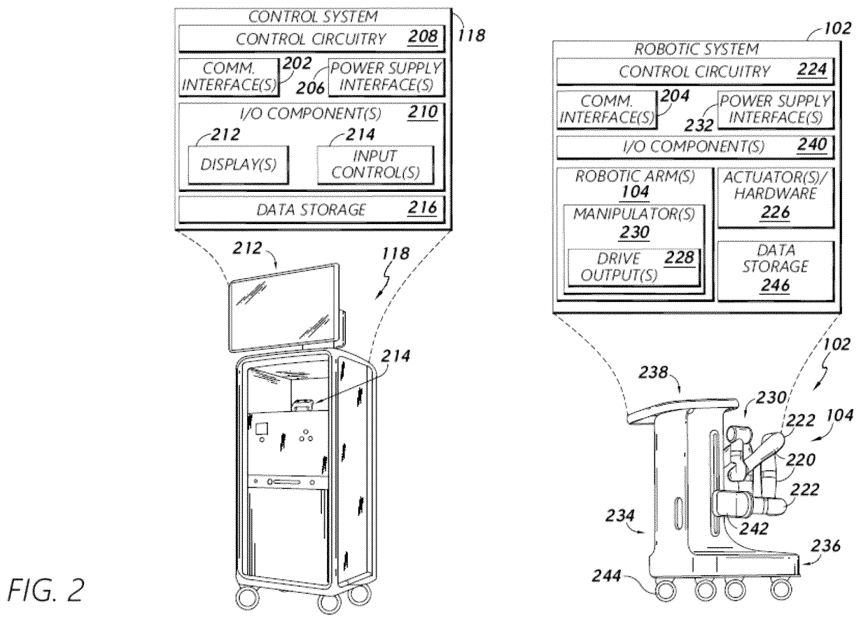

FIG. 2 shows example components of the control system 118 and the robotic system 102 of FIG. 1, according to some implementations. In the examples of FIG. 2, the control system 118 and the robotic system 102 are implemented as a tower and a robotic cart, respectively. However, the control system 118 and robotic system 102 can be implemented in other manners. The control system 118 can be coupled to the robotic system 102 and operate in cooperation therewith to perform a medical procedure. For example, the control system 118 can include communication interface(s) 202 for communicating with communication interface(s) 204 of the robotic system 102 via a wireless or wired connection (such as to control the robotic system 102). In some implementations, the control system 118 may communicate with the robotic system 102 to receive position or sensor data therefrom relating to the position of sensors associated with an instrument or member controlled by the robotic system 102. For example, the control system 118 may communicate with the EM field generator 120 to control generation of an EM field in an area around a patient. The control system 118 can further include one or more power supply interface(s) 206.

The control system 118 can include control circuitry 208 configured to cause one or more components of the medical system 100 to actuate or otherwise control any of the various system components, such as carriages, mounts, arms or positioners, medical instruments, imaging devices, position sensing devices, or sensors, among other examples. Further, the control circuitry 208 can be configured to perform other functions, such as cause display of information, process data, receive input, communicate with other components or devices, or any other function or operation described herein.

The control system 118 can further include one or more input or out (I/O) components 210 configured to assist a physician or others in performing a medical procedure. For example, the one or more I/O components 210 can be configured to receive input or provide output to enable a user to control or navigate the medical instrument 106, the robotic system 102, or other instruments or devices associated with the medical system 100. The control system 118 can include one or more displays 212 to provide, display or otherwise present various information regarding a procedure. For example, the one or more displays 212 can be used to present navigation information including a virtual anatomical model of anatomy with a virtual representation of a medical instrument, image data, or other information. The one or more I/O components 210 can include one or more user input control(s) 214, which can include any type of user input (or output) devices or device interfaces, such as one or more buttons, keys, joysticks, handheld controllers (such as video-game-type controllers), computer mice, trackpads, trackballs, control pads, sensors (such as motion sensors or cameras) that capture hand gestures and finger gestures, touchscreens, toggle (such as button) inputs, or interfaces or connectors therefore. In some implementations, such inputs can be used to generate commands for controlling one or more medical instruments, robotic arms, or other components.

The control system 118 can also include data storage 216 configured to store executable instruments (such as computer-readable instructions) that can be executed by the control circuitry 208 to cause the control circuitry 208 to perform various operations or functionality described herein. In some implementations, the data storage 216 also may store telemetry or runtime data (such as sensor data or image data) generated by the medical system 100 or otherwise captured or acquired during a medical procedure. In some implementations, two or more components of the control system 118 can be electrically or communicatively coupled to each other.

The robotic system 102 can include the one or more robotic arms 104 configured to engage with or control, for example, the medical instrument 106 or other elements or components to perform one or more aspects of a procedure. As shown in FIG. 2, each robotic arm 104 can include multiple segments 220 coupled to joints 222, which can provide multiple degrees of movement or freedom. The robotic system 102 can be configured to receive control signals from the control system 118 to perform certain operations, such as to position one or more of the robotic arms 104 in a particular manner or manipulate an instrument, among other examples. In response, the robotic system 102 can control, using control circuitry 224 thereof, actuators 226 or other components of the robotic system 102 to perform the operations. For example, the control circuitry 224 can control insertion or retraction, articulation, or roll of a shaft of the medical instrument 106 or other instrument by actuating one or more drive outputs 228 of a manipulator 230 (or end-effector) coupled to a base of a robotically-controllable instrument. The drive outputs 228 can be coupled to a drive input on an associated instrument, such as an instrument base of an instrument that is coupled to the associated robotic arm 104. The robotic system 102 also may include one or more power supply interfaces 232.

The robotic system 102 can include a support column 234, a base 236, or a console 238. The console 238 can provide one or more I/O components 240, such as a user interface for receiving user input or a display screen (or a dual-purpose device, such as a touchscreen) to provide the physician or user with preoperative or intraoperative data. The support column 234 can include an arm support 242 (also referred to as a “carriage”) for supporting the deployment of the one or more robotic arms 104. The arm support 242 can be configured to vertically translate along the support column 234. Vertical translation of the arm support 242 allows the robotic system 102 to adjust the reach of the robotic arms 104 to meet a variety of table heights, patient sizes, or physician preferences. The base 236 can include wheel-shaped casters 244 (also referred to as “wheels”) that allow the robotic system 102 to move around the operating room. After reaching the appropriate position, the casters 244 can be immobilized using wheel locks to hold the robotic system 102 in place during the procedure.

The joints 222 of each robotic arm 104 can each be independently-controllable or provide an independent degree of freedom available for instrument navigation. In some implementations, each robotic arm 104 may include seven joints that provide seven degrees of freedom, including “redundant” degrees of freedom. Redundant degrees of freedom can allow robotic arms 104 to be controlled to position their respective manipulators 230 at a specific position, orientation, or trajectory in space using different linkage positions and joint angles. This allows for the robotic system 102 to position or direct a medical instrument from a desired point in space while allowing the physician to move the joints 222 into a clinically advantageous position away from the patient to create greater access, while avoiding collisions.

The one or more manipulators 230 (or end-effectors) can be coupled to an instrument base or handle, which can be attached using a sterile adapter component. The combination of the manipulator 230 and instrument base, as well as any intervening mechanics or couplings (such as the sterile adapter), can be collectively referred to as the manipulator or a manipulator assembly. Manipulators or manipulator assemblies can provide power or control interfaces. Example interfaces may include connectors to transfer pneumatic pressure, electrical power, electrical signals, or optical signals from the robotic arm 104 to an instrument base. Manipulators or manipulator assemblies can be configured to manipulate medical instruments (such as surgical tools) using techniques including, for example, direct drives, harmonic drives, geared drives, belts or pulleys, or magnetic drives, among other examples.

The robotic system 102 can also include data storage 246 configured to store executable instruments (such as computer-readable instructions) that can be executed by the control circuitry 224 to cause the control circuitry 224 to perform various operations or functionality described herein. In some implementations, the data storage 216 also may store telemetry or runtime data (such as sensor data or image data) generated by the medical system 100 or otherwise captured or acquired during a medical procedure. In some implementations, two or more of the components of the robotic system 102 can be electrically or communicatively coupled to each other.

Data storage (including the data storage 216, data storage 246, or other data storage or memory) can include any suitable or desirable type of computer-readable media. For example, computer-readable media can include one or more volatile data storage devices, non-volatile data storage devices, removable data storage devices, or nonremovable data storage devices implemented using any technology, layout, or data structure(s) or protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Functionality described herein can be implemented by the control circuitry 208 of the control system 118 or the control circuitry 224 of the robotic system 102, such as by the control circuitry 208 or 224 executing instructions to cause the control circuitry 208 or 224 to perform the functionality. Control circuitry (including the control circuitry 208, control circuitry 224, or other control circuitry) can include circuitry embodied in a robotic system, control system or tower, instrument, or any other component or device. Control circuitry can include any collection of processors, processing circuitry, processing modules or units, chips, dies (such as semiconductor dies including one or more active or passive devices or connectivity circuitry), microprocessors, micro-controllers, digital signal processors, microcomputers, central processing units, field-programmable gate arrays, programmable logic devices, state machines (such as hardware state machines), logic circuitry, analog circuitry, digital circuitry, or any device that manipulates signals (analog or digital) based on hard coding of the circuitry or operational instructions.

Control circuitry referenced herein can further include one or more circuit substrates (such as printed circuit boards), conductive traces and vias, or mounting pads, connectors, or components. Control circuitry can further include one or more storage devices, which may be embodied in a single device, a plurality of devices, or embedded circuitry of a device. Such data storage can comprise read-only memory, random access memory, volatile memory, non-volatile memory, static memory, dynamic memory, flash memory, cache memory, data storage registers, or any device that stores digital information. In examples in which control circuitry includes a hardware or software state machine, analog circuitry, digital circuitry, or logic circuitry, data storage device(s) or register(s) storing any associated operational instructions can be embedded within, or external to, the circuitry comprising the state machine, analog circuitry, digital circuitry, or logic circuitry.

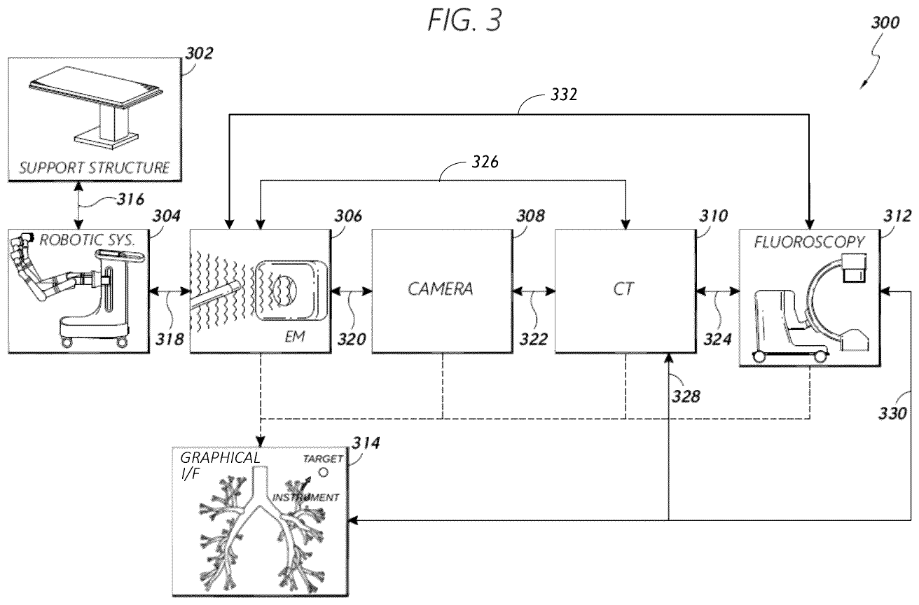

FIG. 3 shows a block diagram of an example localization system 300, according to some implementations. The localization system 300 includes various positioning or imaging systems or modalities 302-312 (also referred to as “subsystems”), which can be implemented to facilitate anatomical mapping, navigation, positioning, or visualization for procedures in accordance with one or more examples. For example, the various systems 302-312 can be configured to provide data for generating an anatomical map, determining a location of an instrument, determining a location of a target, or performing other techniques.

Each of the systems 302-312 can be associated with a respective coordinate space (also referred to as a “position coordinate frame”) or can provide data or information relating to instrument or anatomy locations, wherein registering the various coordinate spaces to one another can allow for integration of the various systems to provide mapping, navigation, or instrument visualization. For example, registering a first modality to a second modality can allow for determined positions in the first modality to be tracked or superimposed on or in a reference frame associated with the second modality, thereby providing layers of positional information that can be combined to provide a robust localization system.

In some implementations, the system 300 may be configured to implement one or more localization or localizing techniques. As used herein, the terms “localization” or “localizing” refer to any processes for determining an instrument position (or location) and orientation (or heading), collectively referred to as the “pose” of the instrument or other element or component, within a given space or environment.

In some implementations, the anatomical space in which a medical instrument can be localized (such as where a position or shape of the instrument is determined or estimated) may be a 2D or 3D portion of a patient's tracheobronchial airways, vasculature, urinary tract, gastrointestinal tract, or any organ or space accessed via lumens. Various modalities can be implemented to provide images, representations, or models of the anatomical space. For example, an imaging modality can be implemented, which can include, for example, X-ray, fluoroscopy, CT, PET, PET-CT, CT angiography, CBCT, 3DRA, SPECT, MRI, OCT, or ultrasound, among other examples. In some implementations, the imaging modality may be used to capture or acquire images of a patient's anatomy during a preoperative phase of a medical procedure. In some other implementations, the imaging modality may be used to capture or acquire images of a patient's anatomy during an intraoperative phase of the medical procedure.

The systems 302-312 can provide information for generating a graphical user interface 314 (also referred to as a “graphical interface (I/F)”) that includes navigation information for navigating an instrument to a target within an anatomy (such as the navigation data 126(A) or 126(B) of FIG. 1). For example, the navigation information may include an anatomical map, an estimated position, orientation, or shape of the instrument, and/or a position of the target. In some implementations, the graphical user interface 314 or other localization information may be displayed to a user, such as a physician, during a medical procedure to assist the user in performing the procedure. For example, a visualization of a tracked instrument can be superimposed on an anatomical map depicted by the graphical user interface 314 based on position or sensor data associated with the tracked medical instrument.

As shown in FIG. 3, the system 300 can include a support structure 302 (such as a surgical bed or other patient positioning or support platform). For example, the support structure 302 includes a planar surface that contacts and supports the patient. In some implementations, the position of the support structure 302 may be known based on data maintained relating to the position of the support structure 302 within the surgical or procedure environment. In some other implementations, the position of the support structure 302 may be sensed or otherwise determined using one or more markers or an appropriate imaging or positioning modality.

The system 300 can further include a robotic system 304 (such as a robotic cart or other device or system including one or more robotic end effectors). In some implementations, the robotic system 304 may be one example of the robotic system 102 of FIGS. 1 and 2. Data relating to the position or state of robotic arms, actuators, or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration, in some implementations, based on robotic system or cart-set-up procedure (which can have any suitable or desirable scheme).

The system 300 can further include an electromagnetic (EM) sensor system 306, which can include an EM field generator (such as the EM field generator 120 of FIG. 1) and one or more EM sensors. An EM sensor can be associated with a portion of an instrument that is tracked or controlled, such as a distal end (or tip) of the instrument or along a length of the instrument or other elongate member (such as a working channel) disposed in a lumen of the instrument. In some implementations, the EM field generator can be mechanically coupled to the support structure 302 or the robotic system 304 such that registration or association 318 between such systems can be known or determined. In some implementations, the registration 318 between the EM sensor system 306 and the robotic system 304 can be determined through forward kinematics or field generator mount transform information. For example, the field generator can be mounted to the support structure 302 such that the position of the field generator can be known relative to the robotic system positioning frame based on a known relationship between the position of the support structure 302 and the robotic system 304. The EM sensor system 306 can provide instrument pose or path information based on sensor readings associated with the instrument.

The system 300 can further include an optical camera system 308 including one or more cameras or other imaging devices configured to generate images of patient anatomy within a visual field thereof (such as real-time image data) during a surgical procedure. In some implementations, registration 320 between the optical camera system 308 and the EM sensor system 306 can be achieved through identification of features having EM sensor data associated therewith, such as a medical instrument tip, in images generated by the optical camera system 308. The registration 320 can further be based at least in part on hand-eye interaction of the physician when viewing real-time camera images while the EM-sensor-equipped endoscope is navigating in the patient anatomy.

The system 300 can further include a computed tomography (CT) imaging system 310 configured to generate CT images of the patient anatomy, which can be performed preoperatively or intraoperatively. The CT imaging system 310 is generally used for scanning a relatively large volume. In some implementations, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the graphical user interface 314 or for path navigation planning.

In some aspects, the CT imaging system 310 may be registered 326 to the EM sensor system 306 through various techniques, such as tool registration or a transformation function, among other examples. In some implementations, a mechanical structure of the CT imaging system 310 can have a known physical transform or relationship with respect to a mounting position of the EM field generator of the EM sensor system 306. Such known relationship can be used to register the CT image space to the EM sensor space. The connection 328 represents a mapping or relationship between the CT imaging system 310 and an anatomical map depicted by a graphical user interface 314.

The system 300 can further include a fluoroscopy imaging system 312 configured to generate tomographic images (such as real-time X-ray images) of the surgical site. The fluoroscopy imaging system 312 is generally used for scanning a smaller volume compared to the CT imaging system 310. In some implementations, the fluoroscopy imaging system 312 may be one example of the imaging system 122 of FIG. 1. For example, the fluoroscopy imaging system 312 may include a CBCT scanner coupled to a C-arm. In some implementations, the fluoroscopy imaging system 312 may be used with a contrast agent introduced into the anatomy to generate image data representing patient anatomy or instrumentation. In some implementations, the fluoroscopy imaging system 312 may be registered 324 to the CT imaging system 310 using any image processing technique suitable for such registration.

In some aspects, the fluoroscopy imaging system 312 may be registered 332 to the EM sensor system 306 through various techniques, such as tool registration or a transformation function, among other examples. In some implementations, a mechanical structure of the fluoroscopy imaging system 312 (such as the C-arm instrumentation) can have a known physical transform or relationship with respect to a mounting position of the EM field generator of the EM sensor system 306. Such known relationship can be used to register the fluoroscopy image space to the EM sensor space. The connection 330 represents a mapping or relationship between the fluoroscopy imaging system 312 and an anatomical map depicted by the graphical user interface 314.

In the example of FIG. 3, the CT imaging system 310 and fluoroscopy imaging system 312 are illustrated as separated systems. However, in some other implementations, a single imaging system may perform the functions of both the CT imaging system 310 and fluoroscopy imaging system 312.

The position, shape, or orientation of an instrument, such as an endoscope, can be determined using any one or more of the systems 302-312, which can facilitate generation of graphical interface data representing the estimated position or shape of the instrument relative to an anatomical map depicted by the graphical user interface 314. The graphical user interface 314 can be displayed on a display device, such as via the control system 118 or robotic system 102, or another device. In some implementations, the graphical user interface 314 also may indicate a position of a target within the anatomy that has been designated for treatment.

Although the systems 302-312 have been described in a particular order, the operations or functions associated therewith can be performed in different orders. In some implementations, the systems 302-312 can be used in different ways. In some other implementations, registration can occur between different systems and modalities.

In some aspects, one or more of the systems 302-312 may be used to generate the graphical user interface 314 preoperatively or determine a location of one or more targets within an anatomical map depicted by the graphical user interface 314 during a preoperative phase of a medical procedure. However, a graphical user interface 314 generated using a preoperative CT scan may not accurately reflect the spatial relationship between a medical instrument and a target during an intraoperative phase. For example, changes in the patient's anatomy or the medical environment can cause the spatial relationship between the instrument and the target to deviate from what is depicted in the graphical user interface 314. Example factors that may cause such deviations include EM distortion, poor registration (or mapping) between the sensor data and the image data, outdated preoperative scans, and anatomical deformations, among other examples. Thus, in some other aspects, one or more of the systems 302-312 may be used to determine a location of a medical instrument or position of a target relative to an anatomical map depicted by the graphical user interface 314 during an intraoperative phase of the medical procedure.

As described with reference to FIG. 3, image data and sensor data are often associated with different coordinate spaces. For example, the image data may describe data points (such as coordinates or vectors) in relation to a coordinate space defined by an imaging system (such as the fluoroscopy imaging system 312) whereas the sensor data may describe data points (such as coordinates or vectors) in relation to a coordinate space defined by a sensing system (such as the EM sensor system 306). To combine the sensor data and the image data on a single frame of reference (such as the graphical user interface 314), the system 300 may register the coordinate space associated with the image data (also referred to as the “image space”) with the coordinate space associated with the sensor data (also referred to as the “sensor space”). As used herein, the term “registration” refers to a mapping between different coordinate spaces. For example, the system 300 (or a registration system associated therewith) may register 332 the fluoroscopy imaging system 312 with the EM sensor system 306 by determining a mapping or spatial transformation that maps any data point in the image space to a respective data point in the sensor space. The system 300 may further use the registration 332 to dynamically update the graphical user interface 314 to reflect the current or actual spatial relationship between the instrument and the target during the intraoperative phase, as depicted by the image data captured via the fluoroscopy imaging system 312 (such as to facilitate real-time navigation).

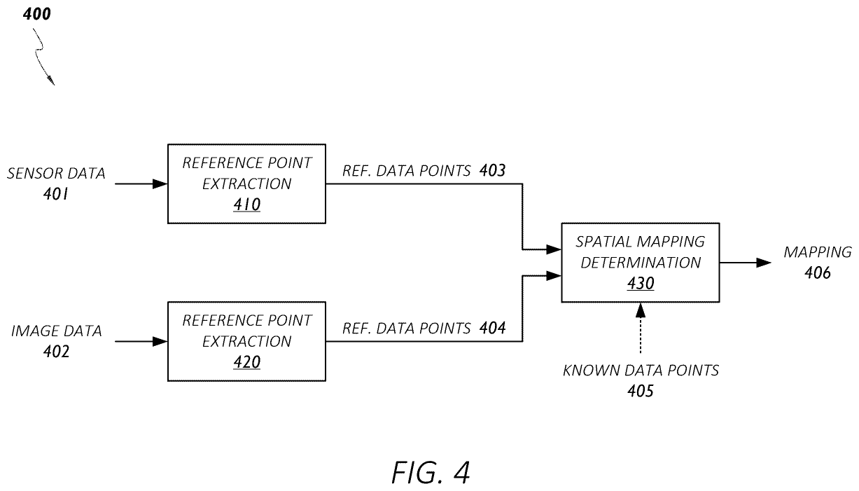

FIG. 4 shows a block diagram of an example registration system 400, according to some implementations. In some implementations, the registration system 400 may be one example of any of the control circuitry 208 or 224 of FIG. 2. The registration system 400 is configured to register a sensor system with an imaging system during an intraoperative phase of a medical procedure to facilitate real-time navigation of a medical instrument within an anatomy. In some implementations, the sensor system and the imaging system may be examples of the EM sensor system 306 and the fluoroscopy imaging system 312, respectively, of FIG. 3.

In some aspects, the registration system 400 may determine a mapping 406 between a coordinate space associated with the sensor system and a coordinate space associated with the imaging system based, at least in part, on sensor data 401 and image data 402 received via the sensor system and the imaging system, respectively. For example, the mapping 406 may be used to transform any data point (such as a coordinate or a vector) in the image space to a respective data point in the sensor space. In some implementations, the sensor data 401 may include position or heading information about one or more EM sensors disposed in an EM field (such as described with reference to FIGS. 1-3). In some implementations, the image data 402 may include one or more tomograms captured by a CBCT scanner (such as described with reference to FIGS. 1-3). Aspects of the present disclosure recognize that some imaging technologies (such as CT or X-rays) may interfere with some sensor technologies (such as EM). In some aspects, the registration system 400 may determine the mapping 406 based on sensor data 401 that is captured or acquired while the imaging system cannot interfere with the underlying sensor technology (such as when a CBCT system is located outside an EM field).

The registration system 400 includes a first reference point extraction component 410, a second reference point extraction component 420, and a spatial mapping determination component 430. The first reference point extraction component 410 is configured to determine one or more reference data points 403 based on the sensor data 401 and the second reference point extraction component 420 is configured to determine one or more reference data points 404 based on the image data 402. As used herein, the term “reference data point” may refer to any object, feature, point, or vector that can be identified or found in both the sensor space and the image space. In some implementations, the reference data points 403 and 404 may include a position of a medical instrument (such as a position of the tip of an endoscope). In some other implementations, the reference data points 403 and 404 may include a heading of the medical instrument (such as an orientation of the tip of the endoscope).

For example, the first reference point extraction component 410 may determine the position and heading of an endoscope (in the sensor space) based on sensor data 401 associated with an EM sensor disposed on or coupled to the tip of the endoscope. The second reference point extraction component 420 may determine the position and heading of the same scope tip (in the image space) through segmentation of the image data 402. As used herein, the term “segmentation” refers to various techniques for partitioning a digital image into groups of voxels (or “image segments”) based on related characteristics or identifying features. Example suitable segmentation techniques include machine learning (ML) models, masking, thresholding, clustering, and edge detection, among other examples. In some implementations, the second reference point extraction component 420 may segment the image data 402 so that the position and heading of the scope tip can be detected or estimated from the corresponding tomograms.

The spatial mapping determination component 430 is configured to determine the mapping 406 based, at least in part, on the reference data points 403 in the sensor space and the reference data points 404 in the image space. In some implementations, the mapping 406 may be a transformation matrix. Aspects of the present disclosure recognize that at least 3 unique reference data points (which may include any combination of coordinates or vectors) are needed to define a unique transformation matrix in 3D space. For example, given 3 unique reference data points, the spatial mapping determination component 430 can determine a transformation matrix that transforms the 3 reference data points in the image space to the 3 reference data points in the sensor space using various known techniques or algorithms. Example suitable algorithms for determining the spatial transformation matrix include singular value decomposition (SVD) and the iterative closest point algorithm (ICP), among other examples.

In some implementations, each of the reference point extraction components 410 and 420 may be configured to extract at least 3 unique reference data points 403 and 404 from the sensor data 401 and the image data 402, respectively. In such implementations, the spatial mapping determination component 430 may determine the mapping 406 using only the reference data points 403 and 404. However, in some other implementations, at least one of the reference point extraction components 410 or 420 may provide fewer than 3 unique reference data points 403 or 404, respectively. For example, the reference data points 403 may include only the position or heading of the instrument in the sensor space and the reference data points 404 may include only the position or heading of the instrument in the image space. In such implementations, the spatial mapping determination component 430 may rely on one or more known data points 405 in the image space and the sensor space to determine the mapping 406.

In some aspects, the known data points 405 may be based on one or more assumptions or known characteristics about the medical system or the surrounding environment. With reference for example to FIG. 1, aspects of the present disclosure recognize that the C-arm 124 of the imaging system 122 is positioned orthogonal to the table 112 (or bed) that supports the patient 110 and the table 112 is generally parallel to the floor. In some implementations, the EM field generator 120 may be a window field generator (WFG) positioned directly beneath (or above) the table 112 so that an open center (or “window”) of the WFG is parallel to, and overlaps, a planar surface of the table 112 (such as the surface of the table 112 on which the patient 110 lies). By positioning the EM field generator 120 and the imaging system 122 at known orientations or tilt angles in relation to the table 112, aspects of the present disclosure can control the relationship between the sensor space and the image space. For example, by positioning the EM field generator 120 parallel to the table 112 and positioning the imaging system 122 orthogonal to the table 112 (such as at a 0° cranial/caudal tilt angle), the image space may be described by a 90- or 180-degree rotation relative to the sensor space. Any discrepancies or offsets in the angle of the table 112 or the tilt-angle of the C-arm 124 can be accounted for by rotating the data set in the sensor space or the image space, for example, to ensure that the two data sets have high correspondence.

Accordingly, various known data points 405 can be inferred or extrapolated from a known spatial relationship between the imaging system and the sensors system. In some implementations, the known data points 405 may include a vector pointing towards the floor of the medical environment. For example, such vector may be orthogonal to the planar surface of the patient bed and the window of the WFG. In some other implementations, the known data points 405 may include a vector pointing towards the head (or toes) of the patient. For example, such vector may be parallel to the planar surface of the patient bend and the window of the WFG. In some implementations, the mapping determination component 430 may determine the mapping 406 based on one or more reference data points 403 and 404 and one or more known data points 405. More specifically, the mapping determination component 430 may determine the mapping 406 based on any combination of 3 (or more) unique data points 403-405.

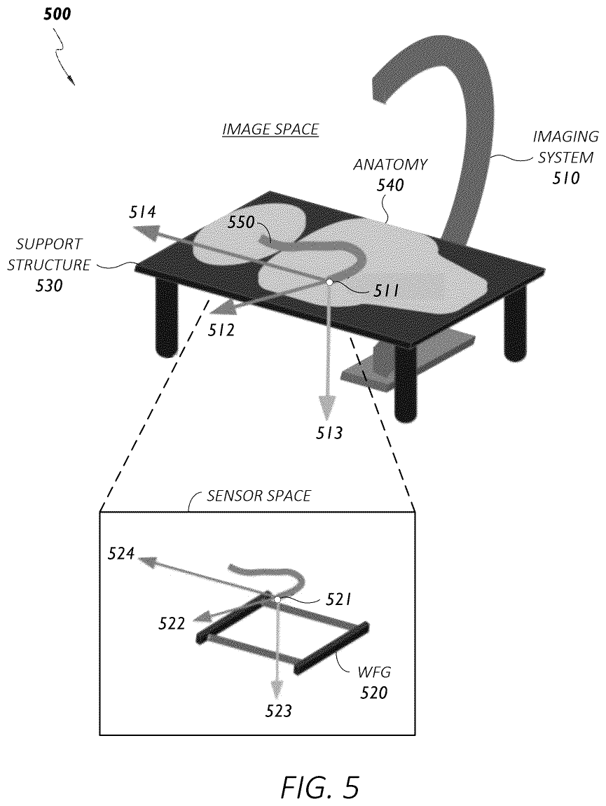

FIG. 5 shows another example medical system 500, according to some implementations. The medical system 500 includes an imaging system 510, a WFG 520, a support structure 530 configured to support a patient anatomy 540, and an endoscope 550 inserted into the patient anatomy 540. In some implementations, the medical system 500 may be one example of the medical system 100 of FIG. 1. With reference for example to FIG. 1, the imaging system 510 may be one example of the imaging system 122, the WFG 520 may be one example of the EM field generator 120, the support structure 530 may be one example of the table 112, and the endoscope 550 may be one example of the medical instrument 106.

In the example of FIG. 5, the WFG 520 is disposed beneath the support structure 530 (as shown in the exploded view) so that a window of the WFG 520 is parallel to a planar surface of the support structure 530 on which the anatomy 540 lies. The distal end (or tip) of the endoscope 550 includes an EM sensor that can interact with an EM field generated by the WFG 520 to produce sensor data indicating a position 521 and a heading 522 of the scope tip in the sensor space (such as the sensor data 401 of FIG. 4). Due to the density of its materials, the endoscope 550 can be segmented from image data captured by the imaging system 510 (such as the image data 402 of FIG. 4). In some implementations, the medical system 500 may determine a position 511 and a heading 512 of the scope tip in the image space based on the segmented image data. In the example of FIG. 5, the data points 511 and 512 describe the same pose of the endoscope 550, but in different coordinate spaces, as the data points 521 and 522. Accordingly, the position 511 of the scope tip in the image space can be mapped to the position 521 of the scope tip in the sensor space, and the heading 512 of the scope tip in the image space can be mapped to the heading 522 of the scope tip in the sensor space.

In some aspects, the medical system 500 may assume that a vector 513 pointing from the tip of the endoscope 550 towards the floor in the image space can be mapped to a vector 523 pointing from the tip of the endoscope 550 towards the floor in the sensor space. As shown in FIG. 5, the vectors 513 and 523 (also referred to as “floor vectors”) are orthogonal to the window of the WFG 520 and the planar surface of the support structure 530. For example, the floor vector 523 can be defined as the vector [0 0 1]^{T} according to a cartesian coordinate system associated with the sensor space. Based on the relative positioning of the imaging system 510 and the WFG 520, the medical system 500 may determine that the floor vector 513 in the image space is rotated 90 degrees relative to the floor vector 523 in the sensor space. Accordingly, the floor vector 513 can be described by the vector [0 1 0]^{T} according to a left-posterior-superior (LPS) coordinate system associated with the image space. Alternatively, the floor vector 513 can be described by the vector [0 −1 0]^{T} according to a right-anterior-inferior (RAI) coordinate system.

In some aspects, the medical system 500 may assume that a vector 514 pointing from the tip of the endoscope 550 towards the head (or toes) of the patient in the image space can be mapped to a vector 524 pointing from the tip of the endoscope 550 towards the head (or toes) of the patient in the sensor space. As shown in FIG. 5, the vectors 514 and 524 (also referred to as “head-to-toe vectors”) are parallel to the window of the WFG 520 and the planar surface of the support structure 530. The head-to-toe vectors 514 and 524 are also orthogonal to the floor vectors 513 and 523, respectively. Thus, the head-to-toe vector 524 can be defined as the vector [0 1 0]^{T} or [0 −1 0]^{T} according to a cartesian coordinate system associated with the sensor space (depending on the orientation of the WFG 520). In some implementations, the medical system 500 may determine whether the head-to-toe vector 524 is [0 1 0]^{T} or [0 −1 0]^{T} based on how the WFG 520 is registered to an image space associated with preoperative CT scans (such as the registration 326 of FIG. 3). The medical system 500 may further assume that the head-to-toe vector 514 in the current image space is rotated 90 degrees relative to the head-to-toe vector 524 in the sensor space. Thus, the head-to-toe vector 514 can be described by the vector [0 0 1]^{T} according to the LPS coordinate system associated with the image space. Alternatively, the head-to-toe vector 514 can be described by the vector [0 0 −1]^{T} according to the RAI coordinate system.

In some implementations, the floor vectors 513 and 523 and head-to-toe vectors 514 and 524 may be examples of the known data points 405 of FIG. 4. As described with reference to FIG. 4, only 3 unique data points are needed to determine a transformation matrix that maps any data points in the image space to respective data points in the sensor space. In some instances, the headings 512 and 522 of the endoscope 550 may overlap with the floor vectors 513 and 523 or the head-to-toe vectors 514 and 524. In such instances, the registration system 400 may still use the floor vectors 513 and 523 and the head-to-toe vectors 514 and 524, together with the positions 511 and 521 of the endoscope 550, to determine the mapping 406. In other words, the floor vectors 513 and 523, head-to-toe vectors 514 and 524, and positions 511 and 521 of the endoscope 550 can always provide a set of 3 unique reference data points even if the headings 512 and 522 overlap with one of the other vectors.

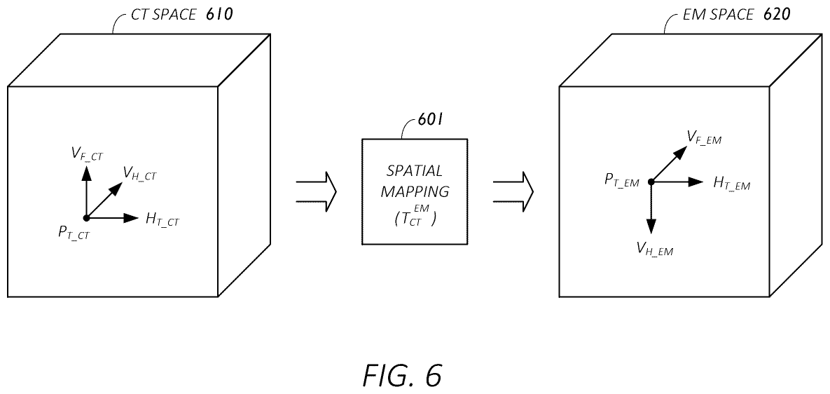

FIG. 6 shows an example mapping 601 between a CT coordinate space 610 and an EM coordinate space 620, according to some implementations. In some implementations, the CT space 610 and the EM space 620 may be examples of the image space and the sensor space, respectively, associated with the imaging system 510 and the WFG 520 of FIG. 5.

The CT space 610 is shown to include a point coordinate P_{T_CT} which forms the origin for a set of vectors H_{T_CT}, V_{F_CT}, and V_{H_CT}. With reference for example to FIG. 5, the point P_{T_CT} may represent the position 511 of the endoscope 550 in the image space, the vector H_{T_CT} may represent the heading 512 of the endoscope 550 in the image space, the vector V_{F_CT} may represent the floor vector 513 in the image space, and the vector V_{H_CT} may represent the head-to-toc vector 514 in the image space. In the example of FIG. 6, the heading vector H_{T_CT} is described by the cartesian coordinates [−1 0 0]^{T}, the floor vector V_{F_CT} is described by the cartesian coordinates [0 1 0]^{T}, and the head-to-toe vector V_{H_CT} is described by the cartesian coordinates [0 0 1]^{T} in the CT space 610.

The EM space 620 is shown to include a point coordinate P_{T_EM} which forms the origin for a set of vectors H_{T_EM}, V_{F_EM}, and V_{H_EM}. With reference for example to FIG. 5, the point P_{T_EM} may represent the position 521 of the endoscope 550 in the sensor space, the vector H_{T_EM} may represent the heading 522 of the endoscope 550 in the sensor space, the vector V_{F_EM} may represent the floor vector 523 in the sensor space, and the vector V_{H_EM} may represent the head-to-toe vector 524 in the sensor space. In the example of FIG. 6, the heading vector H_{T_EM} is described by the cartesian coordinates [−1 0 0]^{T}, the floor vector V_{F_EM} is described by the cartesian coordinates [0 0 1]^{T}, and the head-to-toe vector V_{H_EM} is described by the cartesian coordinates [0 −1 0]^{T} in the EM space 620.

As shown in FIG. 6, the spatial mapping 601 transforms the point coordinate P_{T_CT} and the set of vectors H_{T_CT}, V_{F_CT}, and V_{H_CT} from the CT space 610 to the point coordinate P_{T_EM} and the set of vectors H_{T_EM}, V_{F_EM}, and V_{H_EM}, respectively, in the EM space 620. In some implementations, the spatial mapping 601 may be one example of the mapping 406 of FIG. 4. With reference for example to FIG. 4, the registration system 400 may determine the spatial mapping 601 based on the datapoints P_{T_CT}, H_{T_CT}, V_{F_CT}, V_{H_CT}, P_{T_EM}, H_{T_EM}, V_{F_EM}, and V_{H_EM}. More specifically, the registration system 400 may calculate a transformation matrix T_{CT}^{EM} that transforms the set of data points C=[P_{T_CT}−M_{T_CT} H_{T_CT} V_{F_CT} V_{H_CT}] into the set of data points E=[P_{T_EM}−M_{T_EM} H_{T_EM} V_{F_EM} V_{H_EM}], where:

$$E = {T}_{CT}^{EM} C$$

where M_{T_CT} denotes the centroid of points in the CT space 610 and M_{T_EM} denotes the centroid of points in the EM space 620. Because there is only one known coordinate point in each of the CT space 610 (P_{T_CT}) and the EM space 620 (P_{T_EM}), M_{T_CT}=P_{T_CT} and M_{T_EM}=P_{T_EM}.

The transformation matrix Ta includes a rotation matrix R_{reg} and a translation vector t_{reg}, where T_{CT}^{EM}=(R_{reg} t_{reg}). In some implementations, the registration system 400 may solve for the rotation matrix R_{reg} using singular value decomposition (SVD). For example, the registration system 400 may determine an orthogonal matrix U, a diagonal matrix S, and an orthogonal matrix V, where:

SVD(C E^{T})=U S V^{T}

R_{reg}=V U^{T}

The registration system 400 may further calculate the translation vector t_{reg} based on the point coordinates P_{T_CT} and P_{T_EM}, where:

$${t}_{reg} = {P}_{T \_ EM} - {R}_{reg} {P}_{T \_ CT}$$

As shown in FIG. 6, the rotation matrix R_{reg} merely rotates the vectors H_{T_CT}, V_{F_CT}, and V_{H_CT} 90 degrees around the x-axis in the CT space 610. Thus, as an alternative to SVD, the registration matrix 400 may determine a first rotation matrix R_{reg}¹ that transforms the heading vector H_{T_CT} in the CT space 610 to the heading vector H_{T_EM} in the EM space 620 (assuming the vectors have the same origin). The registration matrix 400 may apply the rotation matrix R_{reg}¹ to the set of data points C=[P_{T_CT} H_{T_CT} V_{F_CT} V_{H_CT}] and determine a second rotation matrix R_{reg}² that rotates the remaining vectors R_{reg}¹V_{F_CT} and R_{reg}¹V_{H_CT} around the heading vector R_{reg}¹H_{T_CT} to transform the vectors R_{reg}¹V_{F_CT} and R_{reg}¹V_{H_CT} into the vectors V_{F_CT} and V_{H_CT}, respectively, in the EM space 620, where R_{reg}=R_{reg}¹R_{reg}².

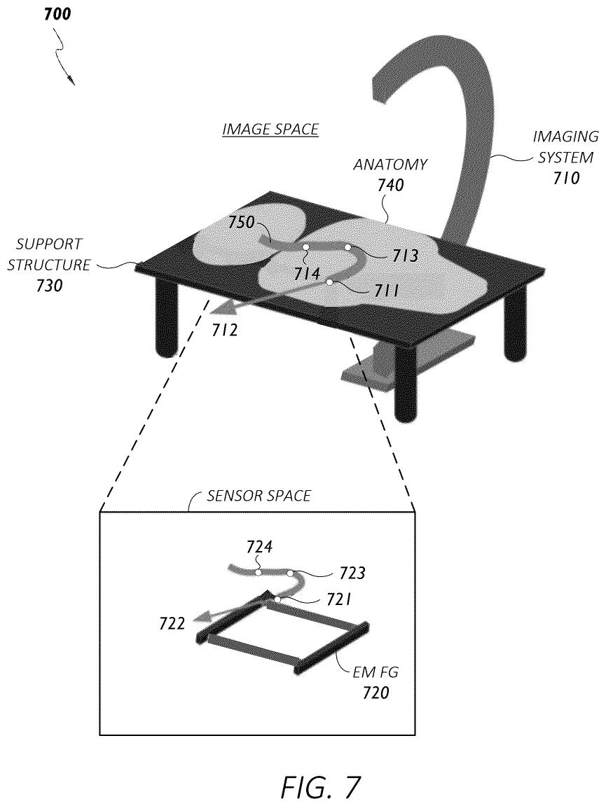

FIG. 7 shows another example medical system 700, according to some implementations. The medical system 700 includes an imaging system 710, an EM field generator (FG) 720, a support structure 730 configured to support a patient anatomy 740, and an endoscope 750 inserted into the patient anatomy 740. In some implementations, the medical system 700 may be one example of the medical system 100 of FIG. 1. With reference for example to FIG. 1, the imaging system 710 may be one example of the imaging system 122, the EM field generator 720 may be one example of the EM field generator 120, the support structure 730 may be one example of the table 112, and the endoscope 750 may be one example of the medical instrument 106.

In the example of FIG. 7, the EM field generator 720 is depicted as a WFG disposed beneath the support structure 730 (as shown in the exploded view) so that a window of the WFG is parallel to a planar surface of the support structure 730 on which the anatomy 740 lies. Alternatively, the EM field generator 720 may be disposed above the support structure 730 (such as in the form of a tabletop field generator). However, in some other implementations, the EM field generator 720 may be any suitable EM field generator capable of producing one or more EM fields in or proximate to the anatomy 740. The distal end of the endoscope 750 includes an EM sensor that can interact with an EM field generated by the EM field generator 720 to produce sensor data indicating a position 721 and/or a heading 722 of the scope tip in the sensor space. In some implementations, the endoscope 750 may further include one or more additional EM sensors disposed at predetermined distances along the length of the shaft. For example, as shown in FIG. 7, the additional EM sensors can produce sensor data indicating positions of predetermined points 723 and 724 along the shaft of the endoscope 750. The sensor data also may indicate headings of the additional EM sensors (not shown for simplicity). In some implementations, the sensor data produced by the EM sensors may be examples of the sensor data 401 of FIG. 4.

Due to the density of its materials, the endoscope 750 can be segmented from image data captured by the imaging system 710 (such as the image data 402 of FIG. 4). In some implementations, the medical system 700 may determine a position 711 and/or a heading 712 of the scope tip in the image space based on the segmented image data. Because the additional EM sensors are disposed at predetermined distances (from the scope tip) along the length of the endoscope 750, the medical system 700 also may determine positions 713 and 714 and/or headings (not shown for simplicity) of the additional EM sensors from the segmented image data. In the example of FIG. 7, the data points 721-724 describe the same pose (and shape) of the endoscope 750, but in different coordinate spaces, as the data points 711-714. Accordingly, the positions 711, 713, and 714 and/or headings of the EM sensors in the image space can be mapped to the positions 721, 723, and 724 and/or headings of the EM sensors, respectively, in the sensor space. Similarly, the heading 712 of the scope tip in the image space can be mapped to the heading 722 of the scope tip in the sensor space.

As described with reference to FIGS. 4 and 6, the medical system 700 may determine a transformation matrix that maps any data point in the image space to a respective data point in the sensor space based on 3 unique reference data points (which can include any combination of point coordinates or vectors). Thus, when the positions and/or headings of at least 3 EM sensors can be detected in each of the sensor space and the image space, the medical system 700 may not require the heading vector of the endoscope 750 to determine the transformation matrix. In some implementations, the medical system 700 may guide a user or physician to collect sensor and image data that captures at least 2 EM sensors. However, if fewer than 2 EM sensors are detected in at least one of the sensor space or the image space, the medical system 700 may rely on one or more known data points (such as the known data points 405 of FIG. 4) to determine the transformation matrix. In some implementations, the known data points may include a floor vector pointing from the tip of the endoscope 750 towards the floor of the medical environment or a head-to-toe vector pointing from the tip of the endoscope 750 towards the head (or toes) of the patient (such as described with reference to FIG. 5).

In some other implementations, the medical system 700 may determine the transformation matrix based on a shape of the endoscope 750. For example, the medical system 700 may estimate the shape of the endoscope 750 in the sensor space based on EM sensor data (such as position and/or heading information) collected from multiple EM sensors disposed along the length of the endoscope 750. The medical system 700 may further estimate the shape of the endoscope 750 in the image space based on segmented image data. In such implementations, the medical system 700 may determine a transformation matrix that transforms multiple points along the estimated shape of the instrument in the image space to corresponding points along the estimated shape of the instrument in the sensor space.

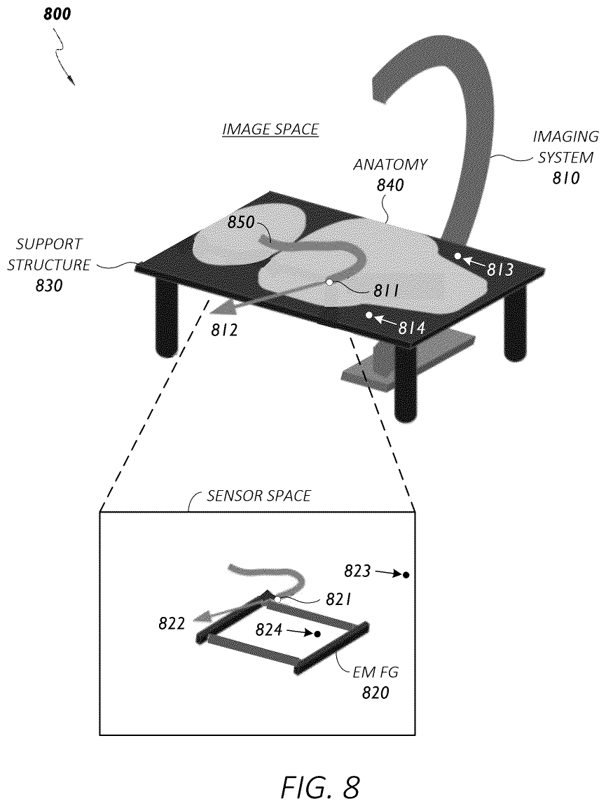

FIG. 8 shows another example medical system 800, according to some implementations. The medical system 800 includes an imaging system 810, an EM field generator (FG) 820, a support structure 830 configured to support a patient anatomy 840, and an endoscope 850 inserted into the patient anatomy 840. In some implementations, the medical system 800 may be one example of the medical system 100 of FIG. 1. With reference for example to FIG. 1, the imaging system 810 may be one example of the imaging system 122, the EM field generator 820 may be one example of the EM field generator 120, the support structure 830 may be one example of the table 112, and the endoscope 850 may be one example of the medical instrument 106.

In the example of FIG. 8, the EM field generator 820 is depicted as a WFG disposed beneath the support structure 830 (as shown in the exploded view) so that a window of the WFG is parallel to a planar surface of the support structure 830 on which the anatomy 840 lies. However, in some other implementations, the EM field generator 820 may be any suitable EM field generator capable of producing one or more EM fields in or proximate to the anatomy 840. The distal end of the endoscope 850 includes an EM sensor that can interact with an EM field generated by the EM field generator 820 to produce sensor data indicating a position 821 and/or a heading 822 of the scope tip in the sensor space. In some implementations, the medical system 800 may further include one or more additional EM sensors disposed at predetermined positions proximate to the anatomy 840. For example, as shown in FIG. 8, the additional EM sensors can be disposed on the support structure 830 to produce sensor data indicating positions of predetermined points 823 and 824 on either side of the anatomy 840. In some implementations, the sensor data produced by the EM sensors may be examples of the sensor data 401 of FIG. 4.

Due to the density of its materials, the endoscope 850 can be segmented from image data captured by the imaging system 810 (such as the image data 402 of FIG. 4). In some implementations, the medical system 800 may determine a position 811 and/or a heading 812 of the scope tip in the image space based on the segmented image data. In some implementations, the additional EM sensors may be radio-opaque so that positions 813 and 814 of the additional EM sensors also can be identified or detected in the segmented image data. In the example of FIG. 8, the data points 811 and 812 describe the same pose of the endoscope 850, but in different coordinate spaces, as the data points 821 and 822. Accordingly, the position 811 of the scope tip in the image space can be mapped to the position 821 of the scope tip in the sensor space, and the heading 812 of the scope tip in the image space can be mapped to the heading 822 of the scope tip in the sensor space. Because the additional EM sensors can be detected in both image space and the sensor space, the positions 813 and 814 of the additional EM sensors in the image space also can be mapped to the positions 823 and 824 of the additional EM sensors, respectively, in the sensor space.

As described with reference to FIGS. 4 and 6, the medical system 800 may determine a transformation matrix that maps any data point in the image space to a respective data point in the sensor space based on 3 unique reference data points (which can include any combination of point coordinates or vectors). Thus, when the positions of at least 3 EM sensors can be detected in each of the sensor space and the image space, the medical system 800 may not require the heading vector of the endoscope 850 to determine the transformation matrix. In some implementations, the positions 813 and 814 of the additional EM sensors may be determined (in the image space) based on two or more 2D images (rather than 3D images) captured by the imaging system 810 at different C-arm angles (such as at an anterior-posterior angle and at a 90 degree rotation therefrom). More specifically, the positions 813 and 814 of the additional EM sensors may be determined based on a rigid transformation from the 2D images to a 3D space (which may be known or predetermined).

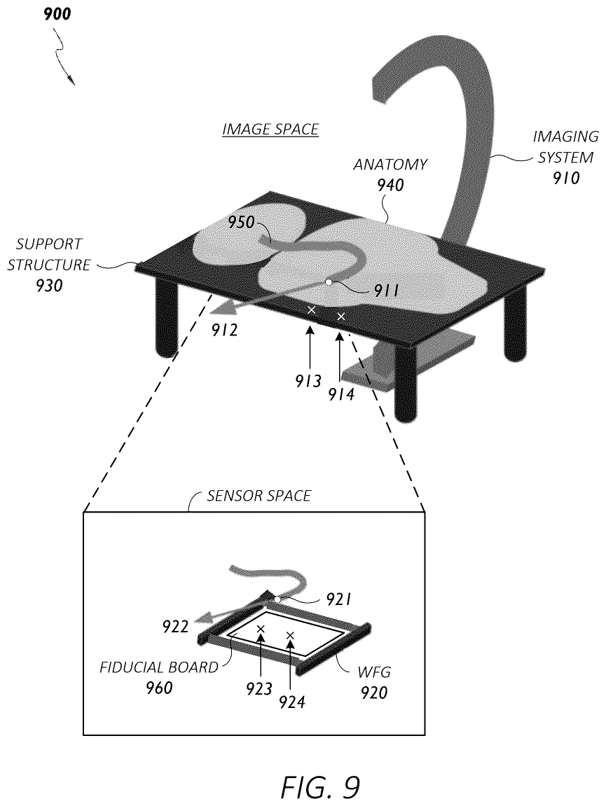

FIG. 9 shows another example medical system 900, according to some implementations. The medical system 900 includes an imaging system 910, an EM field generator (FG) 920, a support structure 930 configured to support a patient anatomy 940, and an endoscope 950 inserted into the patient anatomy 940. In some implementations, the medical system 900 may be one example of the medical system 100 of FIG. 1. With reference for example to FIG. 1, the imaging system 910 may be one example of the imaging system 122, the EM field generator 920 may be one example of the EM field generator 120, the support structure 930 may be one example of the table 112, and the endoscope 950 may be one example of the medical instrument 106.

In the example of FIG. 9, the EM field generator 920 is a WFG disposed beneath the support structure 930 (as shown in the exploded view) so that a window of the WFG is parallel to a planar surface of the support structure 930 on which the anatomy 940 lies. In some implementations, the WFG 920 may be coupled to a fiducial board 960 having a predetermined orientation or position relative to the WFG 920. The fiducial board 960 may include one or more radio-opaque fiducial markers disposed thereon. For example, as shown in FIG. 9, the fiducial board 960 includes two fiducial markers 923 and 924 disposed at predetermined positions relative to the WFG 920. In some implementations, the positions of the fiducial markers 923 and 924 in the sensor space may be examples of the known data points 405 of FIG. 4. The distal end of the endoscope 950 includes an EM sensor that can interact with an EM field generated by the EM field generator 920 to produce sensor data indicating a position 921 and/or a heading 922 of the scope tip in the sensor space. In some implementations, the sensor data produced by the EM sensors may be examples of the sensor data 401 of FIG. 4.

Due to the density of its materials, the endoscope 950 can be segmented from image data captured by the imaging system 910 (such as the image data 402 of FIG. 4). In some implementations, the medical system 900 may determine a position 911 and/or a heading 912 of the scope tip in the image space based on the segmented image data. Because the fiducial markers 923 and 924 are radio-opaque, the positions 913 and 914 of the fiducial markers 923 and 924, respectively, also can be identified or detected in the segmented image data. In the example of FIG. 9, the data points 911 and 912 describe the same pose of the endoscope 950, but in different coordinate spaces, as the data points 921 and 922. Accordingly, the position 911 of the scope tip in the image space can be mapped to the position 921 of the scope tip in the sensor space, and the heading 912 of the scope tip in the image space can be mapped to the heading 922 of the scope tip in the sensor space. Because the relative placement of the fiducial markers 923 and 924 is known in the sensor space, and can be detected in the image space, the positions 913 and 914 of the fiducial markers in the image space also can be mapped to known positions of the fiducial markers 923 and 924, respectively, in the sensor space.

As described with reference to FIGS. 4 and 6, the medical system 900 may determine a transformation matrix that maps any data point in the image space to a respective data point in the sensor space based on 3 unique reference data points (which can include any combination of point coordinates or vectors). However, when multiple fiducial markers lie in the same plane (such as the fiducial markers 923 and 924), the medical system 900 may additionally rely on the position 911 or the heading 912 of the scope tip to determine the transformation matrix. In some implementations, the positions 913 and 914 of the fiducial markers may be determined (in the image space) based on two or more 2D images (rather than 3D images) captured by the imaging system 910 at different C-arm angles (such as at an anterior-posterior angle and at a 90 degree rotation therefrom). More specifically, the positions 913 and 914 of the fiducial markers may be determined based on a rigid transformation from the 2D images to a 3D space (which may be known or predetermined).

In the examples described with reference to FIGS. 5-9, a medical system (or registration system) may register the image space with the sensor space based on the pose of an endoscope having 5 mechanical degrees of freedom (including translation in three orthogonal axes plus yaw and pitch of the scope tip). In actual implementations, the endoscope may have 6 mechanical degrees of freedom (including translation in three orthogonal axes plus yaw, pitch, and roll of the scope tip). However, because the endoscope is a cylindrical instrument, the roll of the scope tip may be difficult to detect in the image space due to the limited resolution of existing imaging technologies. Aspects of the present disclosure recognize that, if the roll of the scope tip can be determined in both the EM space and the image space, a medical system may determine the transformation matrix using only the pose of the scope tip itself. In some implementations, a medical system may determine the roll of the scope tip when a working channel is inserted therein.

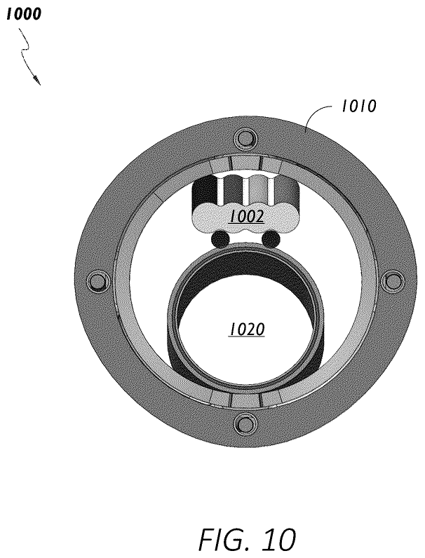

FIG. 10 shows an example medical instrument 1000, according to some implementations. The medical instrument 1000 includes an endoscope 1010 having an inner diameter (or lumen) in which a working channel 1020 is disposed. As shown in FIG. 10, a sensor load 1002 is coupled to the working channel 1020 so that the center of the working channel 1020 is offset relative to the center of the endoscope 1010. As used herein, the term “sensor load” may refer to any electrical wires, circuitry, or other sensor components associated with a sensing system.

As described with reference to FIGS. 1-9, a medical system (such as any of the medical systems 100, 500, 700, 800, or 900 of FIGS. 1, 5, 7, 8, and 9, respectively) may determine the position and heading of the endoscope 1010 based on an EM sensor disposed on the scope tip (not shown for simplicity). In some implementations, the medical system may further detect the offset between the working channel 1020 and the endoscope 1010, and thus the roll of the endoscope 1010, in the sensor space based on one or more sensors disposed on the endoscope 1010 or the working channel 1020. The medical system also may detect the offset between the working channel 1020 and the endoscope 1010 in the image space by segmenting image data captured via an imaging system. As a result, the medical system may determine a transformation matrix between the image space and the sensor space using only the position and orientation (including the yaw, pitch, and roll) of the scope tip in each of the coordinate spaces.

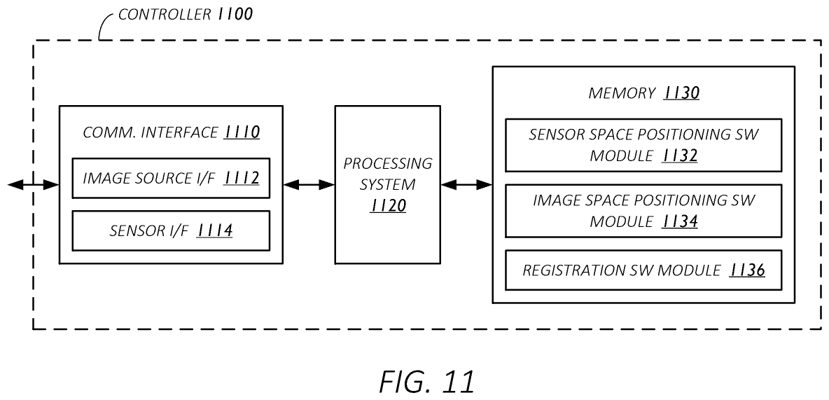

FIG. 11 shows a block diagram of an example controller 1100 for a medical system, according to some implementations. In some implementations, the controller 1100 may be one example of the registration system 400 of FIG. 4. More specifically, the controller 1100 is configured to register a sensor system with an imaging system during an intraoperative phase of a medical procedure to facilitate real-time navigation of an instrument within an anatomy.

The controller 1100 includes a communication interface 1110, a processing system 1120, and a memory 1130. The communication interface 1110 is configured to communicate with one or more components of the medical system. More specifically, the communication interface 1110 includes an image source interface (I/F) 1112 for communicating with one or more image sources (such as the CT imaging system 310 and/or the fluoroscopy imaging system 312 of FIG. 3) and a sensor interface (I/F) 1114 for communicating with one or more sensors (such as the EM sensor system 306 of FIG. 3). In some implementations, the image source I/F 1112 may receive image data captured by an imaging system external to the anatomy while the instrument is disposed within the anatomy. In some implementations, the sensor I/F 1114 may receive sensor data from a sensor disposed on the instrument.

The memory 1130 may include a non-transitory computer-readable medium (including one or more nonvolatile memory elements, such as EPROM, EEPROM, Flash memory, or a hard drive, among other examples) that may store the following software (SW) modules: a sensor space positioning SW module 1132 to determine a position of the instrument in a first coordinate space based on the received sensor data; an image space positioning SW module 1134 to determine a position of the instrument in a second coordinate space based on the received image data; and a registration SW module 1136 to determine a mapping between the first coordinate space and the second coordinate space based at least in part on the position of the instrument in the first coordinate space and the position of the instrument in the second coordinate space. Each of the software modules 1132-1136 includes instructions that, when executed by the processing system 1120, causes the controller 1100 to perform the corresponding functions.

The processing system 1120 may include any suitable one or more processors capable of executing scripts or instructions of one or more software programs stored in the controller 1100 (such as in the memory 1130). For example, the processing system 1120 may execute the sensor space positioning SW module 1132 to determine a position of the instrument in a first coordinate space based on the received sensor data. The processing system 1120 also may execute the image space positioning SW module 1134 to determine a position of the instrument in a second coordinate space based on the received image data. The processing system 1120 may further execute the registration SW module 1136 to determine a mapping between the first coordinate space and the second coordinate space based at least in part on the position of the instrument in the first coordinate space and the position of the instrument in the second coordinate space.

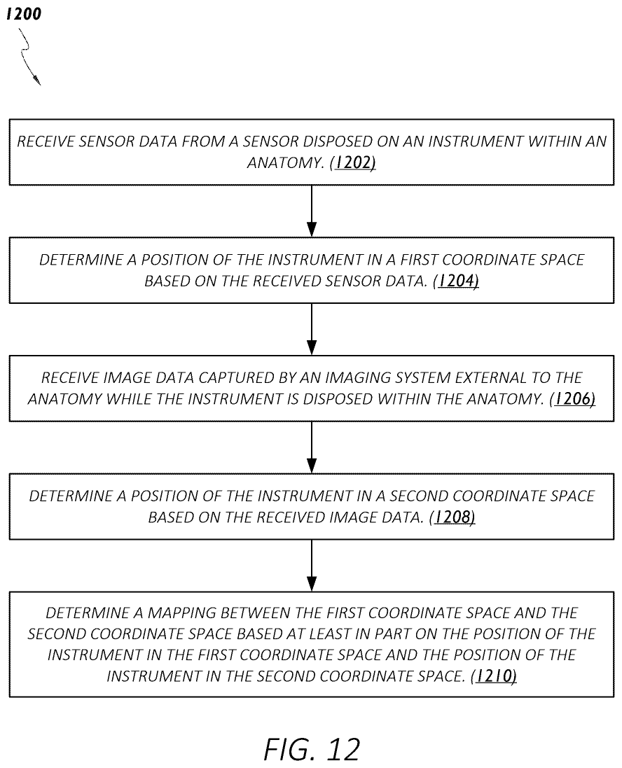

FIG. 12 shows an illustrative flowchart depicting an example operation 1200 for registering an image space with a sensor space, according to some implementations. In some implementations, the example operation 1200 may be performed by a controller for a medical system such as the controller 1100 of FIG. 11 or the registration system 400 of FIG. 4.

The controller may receive sensor data from a sensor disposed on an instrument within an anatomy (1202). The controller may determine a position of the instrument in a first coordinate space based on the received sensor data (1204). The controller may receive image data captured by an imaging system external to the anatomy while the instrument is disposed within the anatomy (1206). In some implementations, the first imaging system may be a CBCT system. The controller may determine a position of the instrument in a second coordinate space based on the received image data (1208). The controller may determine a mapping between the first coordinate space and the second coordinate space based at least in part on the position of the instrument in the first coordinate space and the position of the instrument in the second coordinate space (1210).

In some aspects, the sensor may be an EM sensor disposed within an EM field produced by a field generator coupled to a support structure at a known angle, where the support structure has a planar surface which supports the anatomy. In some implementations, the field generator may a tabletop field generator or a window field generator (WFG) having a planar surface that is parallel to the planar surface of the support structure.

In some aspects, the determining of the mapping between the first coordinate space and the second coordinate space may include estimating, in each of the first coordinate space and the second coordinate space, a first vector orthogonal to the planar surface of the support structure; estimating, in each of the first coordinate space and the second coordinate space, a second vector parallel to the planar surface of the support structure; and determining a transformation matrix that transforms the position of the instrument and the estimated first and second vectors in the second coordinate space to the position of the instrument and the estimated first and second vectors, respectively, in the first coordinate space.

In some implementations, the determining of the transformation matrix may include determining a heading of the instrument in the first coordinate space based on the received sensor data; determining a heading of the instrument in the second coordinate space based on the received image data; and determining a rotation matrix and a translation matrix that transform the position of the instrument, the heading of the instrument, and the first and second vectors in the second coordinate space to the position of the instrument, the heading of the instrument, and the first and second vectors, respectively, in the first coordinate space.

In some aspects, the field generator may be further coupled to a fiducial board including one or more fiducial markers disposed at predetermined positions relative to the field generator. In some implementations, the controller may further determine a position of each of the one or more fiducial markers in the second coordinate space based on the received image data, where the mapping between the first coordinate space and the second coordinate space is further determined based at least in part on the positions of the one or more fiducial markers in the second coordinate space and the predetermined positions of the one or more fiducial markers relative to the field generator.

In some aspects, the controller may further receive additional sensor data from one or more additional sensors proximate to the anatomy; determine positions of the one or more additional sensors in the first coordinate space based on the received additional sensor data; and determine positions of the one or more additional sensors in the second coordinate space based on the received image data, where the mapping between the first coordinate space and the second coordinate space is further determined based at least in part on the positions of the one or more additional sensors in the first coordinate space and the positions of the one or more additional sensors in the second coordinate space. In some implementations, at least one of the one or more additional sensors may be disposed on the instrument. In some other implementations, at least one of the one or more additional sensors may be positioned at a predetermined location external to the anatomy. In some implementations, the positions of the one or more additional sensors in the second coordinate space may be determined based on two or more 2D images captured by the imaging system.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method for registering an image space with a sensor space, comprising:
receiving sensor data from a sensor disposed on an instrument within an anatomy;
determining a position of the instrument in a first coordinate space based on the received sensor data;
receiving image data captured by an imaging system external to the anatomy while the instrument is disposed within the anatomy;
determining a position of the instrument in a second coordinate space based on the received image data; and
determining a mapping between the first coordinate space and the second coordinate space based at least in part on the position of the instrument in the first coordinate space and the position of the instrument in the second coordinate space.

2. The method of claim 1, wherein the imaging system is a cone beam computed tomography (CBCT) system.

3. The method of claim 1, wherein the sensor is an electromagnetic (EM) sensor disposed within an EM field produced by a field generator coupled to a support structure at a known angle, the support structure having a planar surface which supports the anatomy.

4. The method of claim 3, wherein the field generator is a tabletop field generator or a window field generator (WFG) having a planar surface that is parallel to the planar surface of the support structure.

5. The method of claim 3, wherein the determining of the mapping between the first coordinate space and the second coordinate space comprises:
estimating, in each of the first coordinate space and the second coordinate space, a first vector orthogonal to the planar surface of the support structure;
estimating, in each of the first coordinate space and the second coordinate space, a second vector parallel to the planar surface of the support structure; and
determining a transformation matrix that transforms the position of the instrument and the estimated first and second vectors in the second coordinate space to the position of the instrument and the estimated first and second vectors, respectively, in the first coordinate space.

6. The method of claim 5, wherein the determining of the transformation matrix comprises:
determining a heading of the instrument in the first coordinate space based on the received sensor data;
determining a heading of the instrument in the second coordinate space based on the received image data; and
determining a rotation matrix and a translation matrix that transform the position of the instrument, the heading of the instrument, and the first and second vectors in the second coordinate space to the position of the instrument, the heading of the instrument, and the first and second vectors, respectively, in the first coordinate space.

7. The method of claim 3, wherein the field generator is further coupled to a fiducial board including one or more fiducial markers disposed at predetermined positions relative to the field generator.

8. The method of claim 7, further comprising:
determining a position of each of the one or more fiducial markers in the second coordinate space based on the received image data, the mapping between the first coordinate space and the second coordinate space further being determined based at least in part on the positions of the one or more fiducial markers in the second coordinate space and the predetermined positions of the one or more fiducial markers relative to the field generator.

9. The method of claim 1, further comprising:
receiving additional sensor data from one or more additional sensors proximate to the anatomy;
determining positions of the one or more additional sensors in the first coordinate space based on the received additional sensor data; and
determining positions of the one or more additional sensors in the second coordinate space based on the received image data, the mapping between the first coordinate space and the second coordinate space further being determined based at least in part on the positions of the one or more additional sensors in the first coordinate space and the positions of the one or more additional sensors in the second coordinate space.

10. The method of claim 9, wherein at least one of the one or more additional sensors is disposed on the instrument.

11. The method of claim 9, wherein at least one of the one or more additional sensors is positioned at a predetermined location external to the anatomy.

12. The method of claim 9, wherein the positions of the one or more additional sensors in the second coordinate space are determined based on two or more two-dimensional (2D) images captured by the imaging system.

13. A controller for a medical system, comprising:
a processing system; and
a memory storing instructions that, when executed by the processing system, cause the controller to:
receive sensor data from a sensor disposed on an instrument within an anatomy;
determine a position of the instrument in a first coordinate space based on the received sensor data;
receive image data captured by an imaging system external to the anatomy while the instrument is disposed within the anatomy;
determine a position of the instrument in a second coordinate space based on the received image data; and
determine a mapping between the first coordinate space and the second coordinate space based at least in part on the position of the instrument in the first coordinate space and the position of the instrument in the second coordinate space.

14. The controller of claim 13, wherein the imaging system is a cone beam computed tomography (CBCT) system.

15. The controller of claim 13, wherein the sensor is an electromagnetic (EM) sensor disposed within an EM field produced by a field generator coupled to a support structure at a known angle, the support structure having a planar surface which supports the anatomy.

16. The controller of claim 15, wherein the field generator is a tabletop field generator or a window field generator (WFG) having a planar surface that is parallel to the planar surface of the support structure.

17. The controller of claim 15, wherein the determining of the mapping between the first coordinate space and the second coordinate space comprises:
estimating, in each of the first coordinate space and the second coordinate space, a first vector orthogonal to the planar surface of the support structure;
estimating, in each of the first coordinate space and the second coordinate space, a second vector parallel to the planar surface of the support structure; and
determining a transformation matrix that transforms the position of the instrument and the estimated first and second vectors in the second coordinate space to the position of the instrument and the estimated first and second vectors, respectively, in the first coordinate space.

18. The controller of claim 17, wherein the determining of the transformation matrix comprises:
determining a heading of the instrument in the first coordinate space based on the received sensor data;
determining a heading of the instrument in the second coordinate space based on the received image data; and
determining a rotation matrix and a translation matrix that transform the position of the instrument, the heading of the instrument, and the first and second vectors in the second coordinate space to the position of the instrument, the heading of the instrument, and the first and second vectors, respectively, in the first coordinate space.

19. The controller of claim 15, wherein the field generator is further coupled to a fiducial board including one or more fiducial markers disposed at predetermined positions relative to the field generator, execution of the instructions further causing the controller to:
determine a position of each of the one or more fiducial markers in the second coordinate space based on the received image data, the mapping between the first coordinate space and the second coordinate space further being determined based at least in part on the positions of the one or more fiducial markers in the second coordinate space and the predetermined positions of the one or more fiducial markers relative to the field generator.

20. The controller of claim 12, wherein execution of the instructions further causes the controller to:
receive additional sensor data from one or more additional sensors proximate to the anatomy;
determine positions of the one or more additional sensors in the first coordinate space based on the received additional sensor data; and
determine positions of the one or more additional sensors in the second coordinate space based on the received image data, the mapping between the first coordinate space and the second coordinate space further being determined based at least in part on the positions of the one or more additional sensors in the first coordinate space and the positions of the one or more additional sensors in the second coordinate space.
